SEQUENCE LISTING (57 sequences)

SEQ ID NO: 1 - DNA, 22 bp, synthetic construct
  source 1..22: mol_type = other DNA; note = synthetic primer
gaaaggcctcatcagggctcag

SEQ ID NO: 2 - DNA, 27 bp, synthetic construct
  source 1..27: mol_type = other DNA; note = synthetic primer
tcctcactgggaaacatgaggaatgac

SEQ ID NO: 3 - DNA, 81 bp, Homo sapiens
  source 1..81: mol_type = genomic DNA
gaaaggcctcatcagggctcagaaaggtgacccaagcagctgggaacacacggggtcattcctcatgtttcccagtgagga

SEQ ID NO: 4 - DNA, 81 bp, Homo sapiens; identical to SEQ ID NO: 3
  source 1..81: mol_type = genomic DNA

SEQ ID NO: 5 - DNA, 81 bp, Homo sapiens
  source 1..81: mol_type = genomic DNA
gaaaggcctcatcagggctcagaaaggtgacccaagcagctgggaacacatggggtcattcctcatgtttcccagtgagga

SEQ ID NO: 6 - DNA, 81 bp, Homo sapiens; identical to SEQ ID NO: 3
  source 1..81: mol_type = genomic DNA

SEQ ID NO: 7 - DNA, 81 bp, Homo sapiens; identical to SEQ ID NO: 3
  source 1..81: mol_type = genomic DNA

SEQ ID NO: 8 - DNA, 81 bp, Homo sapiens; identical to SEQ ID NO: 3
  source 1..81: mol_type = genomic DNA

SEQ ID NO: 9 - DNA, 81 bp, Homo sapiens; identical to SEQ ID NO: 3
  source 1..81: mol_type = genomic DNA

SEQ ID NO: 10 - DNA, 81 bp, Homo sapiens; identical to SEQ ID NO: 5
  source 1..81: mol_type = genomic DNA

SEQ ID NO: 11 - DNA, 81 bp, Homo sapiens; identical to SEQ ID NO: 3
  source 1..81: mol_type = genomic DNA

SEQ ID NO: 12 - DNA, 25 bp, synthetic construct
  source 1..25: mol_type = other DNA; note = synthetic probe
ggtgacccaagcagctgggaacaca

SEQ ID NO: 13 - DNA, 26 bp, synthetic construct
  source 1..26: mol_type = other DNA; note = synthetic primer
catgaacgtcctggattctgtcactc

SEQ ID NO: 14 - DNA, 33 bp, synthetic construct
  source 1..33: mol_type = other DNA; note = synthetic primer
tcactctctgtcttcctctcaaggaatttctac

SEQ ID NO: 15 - DNA, 26 bp, synthetic construct
  source 1..26: mol_type = other DNA; note = synthetic primer
gccatgaacgtcctggattctgtcac

SEQ ID NO: 16 - DNA, 25 bp, synthetic construct
  source 1..25: mol_type = other DNA; note = synthetic probe
caggctccctgaataggcaggtgtg

SEQ ID NO: 17 - DNA, 359 bp, Homo sapiens
  source 1..359: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtgacagaatccaggacgttcatg

SEQ ID NO: 18 - DNA, 359 bp, Homo sapiens; identical to SEQ ID NO: 17
  source 1..359: mol_type = genomic DNA

SEQ ID NO: 19 - DNA, 359 bp, Homo sapiens
  source 1..359: mol_type = genomic DNA
tcctcactgggaaacatgaggaatgaccccatgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtgacagaatccaggacgttcatg

SEQ ID NO: 20 - DNA, 357 bp, Homo sapiens
  source 1..357: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcgcagtataagggggttgacacacctgcctattcagggagcctgggtgctcatttcagaaatgtagaaactgaggctcctttcgtacatgtagaaattccttgagaggaagacagagtgacagaatccaggacgttcatg

SEQ ID NO: 21 - DNA, 359 bp, Homo sapiens; identical to SEQ ID NO: 17
  source 1..359: mol_type = genomic DNA

SEQ ID NO: 22 - DNA, 359 bp, Homo sapiens; identical to SEQ ID NO: 17
  source 1..359: mol_type = genomic DNA

SEQ ID NO: 23 - DNA, 359 bp, Homo sapiens; identical to SEQ ID NO: 17
  source 1..359: mol_type = genomic DNA

SEQ ID NO: 24 - DNA, 359 bp, Homo sapiens; identical to SEQ ID NO: 19
  source 1..359: mol_type = genomic DNA

SEQ ID NO: 25 - DNA, 359 bp, Homo sapiens; identical to SEQ ID NO: 17
  source 1..359: mol_type = genomic DNA

SEQ ID NO: 26 - DNA, 340 bp, Homo sapiens
  source 1..340: mol_type = genomic DNA
tcctcactgggaaacatgaggaatgaccccatgtgttcccagctgcttgggtcaccattctgagtcctgatgaggcctttcccgatggattcccctgacagatcctatgtaaggacctgtggtgcaatcccctgcaatcctacaagaggatgaagccacctgaagagggaacagagatttcaggtgagctgttcagttggaactgaagctttttgatccccaggataaggaggttgacacacctgcctattcagggagcctggaggctcatttcagaaatgtagaaattgagcctcctttcatacatgtagaaattccttgagaggaagacagagtgtga

SEQ ID NO: 27 - DNA, 339 bp, Homo sapiens
  source 1..339: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtga

SEQ ID NO: 28 - DNA, 339 bp, Homo sapiens; identical to SEQ ID NO: 27
  source 1..339: mol_type = genomic DNA

SEQ ID NO: 29 - DNA, 339 bp, Homo sapiens
  source 1..339: mol_type = genomic DNA
tcctcactgggaaacatgaggaatgaccccatgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtga

SEQ ID NO: 30 - DNA, 339 bp, Homo sapiens
  source 1..339: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcgcagtataagggggttgacacacctgcctattcagggagcctgggtgctcatttcagaaatgtagaaactgaggctcctttcgtacatgtagaaattccttgagaggaagacagagtgaca

SEQ ID NO: 31 - DNA, 339 bp, Homo sapiens; identical to SEQ ID NO: 27
  source 1..339: mol_type = genomic DNA

SEQ ID NO: 32 - DNA, 339 bp, Homo sapiens; identical to SEQ ID NO: 27
  source 1..339: mol_type = genomic DNA

SEQ ID NO: 33 - DNA, 339 bp, Homo sapiens; identical to SEQ ID NO: 27
  source 1..339: mol_type = genomic DNA

SEQ ID NO: 34 - DNA, 339 bp, Homo sapiens; identical to SEQ ID NO: 29
  source 1..339: mol_type = genomic DNA

SEQ ID NO: 35 - DNA, 339 bp, Homo sapiens; identical to SEQ ID NO: 27
  source 1..339: mol_type = genomic DNA

SEQ ID NO: 36 - DNA, 362 bp, Homo sapiens
  source 1..362: mol_type = genomic DNA
tcctcactgggaaacatgaggaatgaccccatgtgttcccagctgcttgggtcaccattctgagtcctgatgaggcctttcccgatggattcccctgacagatcctatgtaaggacctgtggtgcaatcccctgcaatcctacaagaggatgaagccacctgaagagggaacagagatttcaggtgagctgttcagttggaactgaagctttttgatccccaggataaggaggttgacacacctgcctattcagggagcctggaggctcatttcagaaatgtagaaattgagcctcctttcatacatgtagaaattccttgagaggaagacagagtgtgacagaatccaggacattcatggc

SEQ ID NO: 37 - DNA, 361 bp, Homo sapiens
  source 1..361: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtgacagaatccaggacgttcatggc

SEQ ID NO: 38 - DNA, 361 bp, Homo sapiens; identical to SEQ ID NO: 37
  source 1..361: mol_type = genomic DNA

SEQ ID NO: 39 - DNA, 361 bp, Homo sapiens
  source 1..361: mol_type = genomic DNA
tcctcactgggaaacatgaggaatgaccccatgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcccagtataagggggttgacacacctgcctattcagggagcctgggtgctcatttcagaaatgtagaaattgaggctcctttcgtacatgtagaaattccttgagaggaagacagagagtgacagaatccaggacgttcatggc

SEQ ID NO: 40 - DNA, 359 bp, Homo sapiens
  source 1..359: mol_type = genomic DNA
tcctcactgggaaacatgaggaatgaccccgtgtgttcccagctgcttgggtcacctttctgagccctgatgaggcctttcccgattgagtcccctgacagatcctatgtaaggacctgtggcgcaatcccctgcaatactacaagaggatgaagccacctgaagagggaacagagacgtcaggtgagccgttagttggcactggagctgtttgatgcgcagtataagggggttgacacacctgcctattcagggagcctgggtgctcatttcagaaatgtagaaactgaggctcctttcgtacatgtagaaattccttgagaggaagacagagtgacagaatccaggacgttcatggc

SEQ ID NO: 41 - DNA, 361 bp, Homo sapiens; identical to SEQ ID NO: 37
  source 1..361: mol_type = genomic DNA

SEQ ID NO: 42 - DNA, 361 bp, Homo sapiens; identical to SEQ ID NO: 37
  source 1..361: mol_type = genomic DNA

SEQ ID NO: 43 - DNA, 361 bp, Homo sapiens; identical to SEQ ID NO: 37
  source 1..361: mol_type = genomic DNA

SEQ ID NO: 44 - DNA, 361 bp, Homo sapiens; identical to SEQ ID NO: 39
  source 1..361: mol_type = genomic DNA

SEQ ID NO: 45 - DNA, 361 bp, Homo sapiens; identical to SEQ ID NO: 37
  source 1..361: mol_type = genomic DNA

SEQ ID NO: 46 - DNA, 24 bp, synthetic construct
  source 1..24: mol_type = other DNA; note = synthetic primer
gtagctccataatcagaagatgcg

SEQ ID NO: 47 - DNA, 27 bp, synthetic construct
  source 1..27: mol_type = other DNA; note = synthetic primer
attcgctctactccaaccacataatag

SEQ ID NO: 48 - DNA, 434 bp, Homo sapiens
  source 1..434: mol_type = genomic DNA
gtagctccataatcagaagatgcgctaccgtacacaacggcggtttaaactttgtagacctcacgcggctgctcaggtcttgcgcatcccatacttcacatggacggaaagctgtgggtttgtatcgataagccgtaggttcctaggatcgagtcacccaaacaacttcgagagctcttctcaccaaccctttgaactccgaaatgccgtgaacacatccccttagttggcattggtctactaaccgagtatcgtacaaagcgagacaggtctggcccctactgcttggatgcgaactaacagtgttcctatatccgtttatactgtggtcgatttcgaaccgatatctcaaagagagcgccggcagagccttagtatcggaactggcgagtgtgtttgggtgaggactattatgtggttggagtagagcgaat

SEQ ID NO: 49 - DNA, 26 bp, synthetic construct
  source 1..26: mol_type = other DNA; note = synthetic probe
tccgatactaaggctctgccggcgct

SEQ ID NO: 50 - DNA, 24 bp, synthetic construct
  source 1..24: mol_type = other DNA; note = synthetic primer
cctgaggatgccacagtgagacac

SEQ ID NO: 51 - DNA, 28 bp, synthetic construct
  source 1..28: mol_type = other DNA; note = synthetic primer
acttctgtctactgtcggactctacagg

SEQ ID NO: 52 - DNA, 22 bp, synthetic construct
  source 1..22: mol_type = other DNA; note = synthetic primer
gtggcccatttaggcccaactc

SEQ ID NO: 53 - DNA, 24 bp, synthetic construct
  source 1..24: mol_type = other DNA; note = synthetic primer
ggcccagctttgacttgagaacag

SEQ ID NO: 54 - DNA, 22 bp, synthetic construct
  source 1..22: mol_type = other DNA; note = synthetic primer
gctgggtctacaggcacaactg

SEQ ID NO: 55 - DNA, 24 bp, synthetic construct
  source 1..24: mol_type = other DNA; note = synthetic probe
tctggagggtccactgtgaggcag

SEQ ID NO: 56 - DNA, 24 bp, synthetic construct
  source 1..24: mol_type = other DNA; note = synthetic probe
ccatttccaggcctagtgcctgcc

SEQ ID NO: 57 - DNA, 24 bp, synthetic construct
  source 1..24: mol_type = other DNA; note = synthetic probe
tgcctgttgaggctgctggcaggc

(19) United States
(12) Patent Application Publication
(10) Pub. No.: US 2026/0168014 A1
(43) Pub. Date: Jun. 18, 2026

(54) DIGITAL AMPLIFICATION METHOD FOR QUANTIFYING AND/OR DETERMINING INTEGRITY OF HUMAN MALE DNA

(71) Applicant: QIAGEN GmbH, Hilden (DE)

(72) Inventors: Miroslav VRANES, Hilden (DE); Mario SCHERER, Hilden (DE); Stefan Otto CORNELIUS, Hilden (DE); Ralf PEIST, Hilden (DE); Margaretha WILLUWEIT, Hilden (DE)

(21) Appl. No.: 19/124,861

(22) PCT Filed: Oct. 24, 2023
(86) PCT No.: PCT/EP2023/079631

§ 371 Date: Apr. 26, 2025

(30) Foreign Application Priority Data
Oct. 27, 2022 (EP) 22204109.7

(51) Int. Cl. C12Q 1/6851

(52) U.S. Cl. CPC C12Q 1/6851

(57) ABSTRACT

According to a first aspect of the present invention, a digital amplification method is provided for detecting and quantifying DNA and for determining degradation and/or integrity of the DNA, in particular male DNA in a sample, wherein the method comprises the step of digital amplification of at least two regions on at least one locus within the Y-chromosome.

### FIELD OF THE INVENTION

The present invention is in the field of molecular biology, diagnostics, more particularly in the field of analytical and forensic sciences. The invention is further in the field of nucleic acid amplification and quantification, more particularly in the field of DNA quantification.

### BACKGROUND OF THE INVENTION

DNA-based detection methods are of rising importance in many fields, for example in the field of forensics. Human DNA isolated from various sources has to be assessed in terms of quantity, quality and integrity prior to further forensic testing, e.g. short tandem repeat (STR) analyses or next-generation-sequencing. Since these methods require a defined range of input DNA amount and template quality in order to perform accurately, DNA quantification and integrity assessment are important steps within the forensic workflow.

Further, evidence collection in sexual assault is crucial for the prosecution of sexual offenders. Physical evidence of semen often serves as the criterion for confirming the sexual crime. However, isolation and quantification of DNA from sexual assault samples is often challenging due to the presence of DNA molecules from both, the female victim as well as the male attacker. In a typical sample, the amount of female DNA exceeds the amount of male DNA by several orders of magnitude. Furthermore, evidence is often not collected immediately after the sexual crime, but hours or days later, which can lead to isolation of partially or completely degraded male DNA. Degraded male DNA can lead to loss of amplification of longer amplicons, or even to a complete failure of Y-chromosome STR (Y-STR) analysis.

The determination of the quantity of DNA recovered from forensic samples as well as other samples is a critical step in the overall DNA typing process, but also in the detection of DNA in various other fields of science. A narrow range of input DNA from 0.5 to 2 ng is often needed to produce optimal results with for example multiplex DNA typing kits. Therefore, in order to ensure that a positive result is a positive result and/or a negative result is a negative result due to the absence of DNA, quantification of DNA is of absolute importance. Furthermore, the quality of standards for forensic DNA testing laboratories requires human-specific DNA quantification. This is due to isolation techniques that can recover human DNA as well as bacterial and other exogenous DNA. A number of procedures have been developed to permit quantification of human-specific DNA including start-blot techniques, liquid based hybridization assays and real-time polymerase chain reaction (PCR). Currently, real-time PCR is the dominant technique due to its wide dynamic range and ease of automation. However, for the male DNA quantification by qPCR a standard curve is needed to calculate DNA amount of the sample. Usually the standard curve is prepared by a serial dilution by the user. The preparation of the standard curve is critical for a precise DNA quantification of samples. Depending on the user's skills and quality of the used equipment for standard curve preparation the process of DNA dilution and preparation of the DNA standard curve itself can be very tedious, laborious and error prone which can lead to wrong quantification results and a complete failure of subsequent Y-STR analysis.

Furthermore, methods using digital PCR to quantify human DNA have been limited due to the availability of different dye channels making it impossible to higher multiplex methods to assess DNA quantity and DNA integrity at the same time.

The modern short tandem repeat (STR) kits have become much more sensitive and can obtain good results even using low amounts of DNA. Therefore, there is a desire for a method, kit and nucleic acid region that allows precise and accurate quantification of human male DNA even in low concentrated samples.

There are certain quantification and detection kits already available. One such kit is the Quantifiler Human Kit (Applied Biosystems) another kit is Quantifiler Duo Kit (Applied Biosystems) another kit is the Plexor HY Real-Time PCR Quantification Kit (Promega). Both the Quantifiler Duo Kit and the Plexor HY Kit target an autosomal and a gonosomal (Y-chromosome) target on the genome.

However, the kits currently on the market present some drawbacks. According to LaSalle et al. (Forensic Science International: Genetics, (2011) 5:185-193) the Quantifiler Kits are more accurate in the quantification but have a lower dynamic range as the Plexor HY. The Plexor HY offers a higher dynamic range due to the amplification of a multicopy target, but a lower accuracy. This lower accuracy can be attributed to the multicopy target. If less than the full set of 20 copies on a genome amplify, because of, for example, instability in the target copy number, than the ratio between the amplification between autosomal and gonosomal (Y) target may vary. The dynamic range of the Plexor HY kit is slightly better than that of the other kit (LaSalle et al., Forensic Science International: Genetics, (2011) 5:185-193). In a statistical comparison, it has been demonstrated a significant difference between the two kits (LaSalle et al., Forensic Science International: Genetics, (2011) 5:185-193).

Another important parameter in forensics is the degradation grade of the DNA that has to be analyzed. Since the amplicon size of the Quantifiler Human and Plexor HY vary from 62 to 133 base pairs (bp), significant differences might be expected when the kits are applied to degraded DNA. Also, inhibitors must be taken into account. It may well be that DNA is present in the reaction no result is obtained due to the presence of inhibitory substances.

In cases of sexual assault samples, a quantification of the DNA is challenging, due to the presence of DNA molecules from both, the female victim as well as the male attacker. Furthermore, in a typical sample, the amount of female DNA exceeds the amount of male DNA by several orders of magnitude. Thus, a sensitive and precise male specific DNA quantification method which can accurately detect and quantify male DNA even in a high background of female DNA, is therefore of great interest.

Reported herein is a digital PCR-based (dPCR-based) DNA quantification system that is highly sensitive and precise to detect low amounts of male DNA in a high background of female DNA and assess in parallel the male DNA degradation and/or integrity of the male DNA. It shall be highlighted here that, besides the system/method described in WO2018054783A1, there is no other system/method known yet, which can be used to assess integrity of male DNA in a mixed sample comprising male and female DNA, wherein the amount of female DNA strongly exceeds the amount of male DNA. However, the system/method described in WO2018054783A1 is based on qPCR with all its drawbacks as outlined above.

### SUMMARY OF THE INVENTION

The invention relates to a digital amplification method for detecting and quantifying DNA and for determining degradation and/or integrity of the DNA, in particular male DNA in a sample, wherein the method comprises the step of digital amplification of at least two regions on at least one locus within the Y-chromosome.

In several embodiments of the invention, the at least one locus within the Y-chromosome may be a single copy and/or a multicopy locus (MCL-Y).

In particular, the invention relates to a method for i) detecting DNA, ii) quantifying DNA, and iii) determining the integrity and/or degradation status of DNA, preferably male DNA, in a sample comprising said male DNA, wherein the method comprises the steps of:
a) amplifying at least two regions on at least one locus within the Y-chromosome, wherein the amplification is done by digital PCR,
b) detecting the at least two amplification products through the use of at least two probes,
c) quantifying the amount of the at least two amplification products, and
d) determining the integrity and/or degradation status of the DNA of the sample by calculating a degradation index,
   wherein at least one of the at least two probes binds to one of the at least two amplification products and at least one other probe of the at least two probes binds to another one of the at least two amplification products, and wherein at least one of the at least two amplification products is longer than another one of the at least two amplification products.

The term “at least one” as used in the present invention refers to 1, 2, 3, or more. For example, at least one region or at least one locus is meant to include 1, 2, 3, or more regions or loci, respectively. Similarly, “at least two” as used herein refers to 2, 3, 4 or more. For example, at least two regions or at least two loci is meant to include 2, 3, 4 or more regions or loci, respectively.

In one embodiment of the invention, the at least two regions being amplified are localized on one locus within the Y-chromosome, but are either not overlapping with each other or at least two of the two or more regions are not overlapping.

In another embodiment of the invention, the at least two regions being amplified are localized on at least two loci within the Y-chromosome. Preferably, the at least two loci within the Y-chromosome are single copy and/or multicopy loci or at least one single copy locus and at least one multicopy locus (MCL-Y) within the Y-chromosome.

In yet another embodiment of the invention, the at least two regions being amplified are localized on one locus within the Y-chromosome and are overlapping with each other.

In one embodiment of the invention, the amplification step is performed using at least one primer selected from one of the groups consisting of:
a. SEQ ID NO. 1 and SEQ ID NO. 2,
b. a reverse complement of SEQ ID NO. 1 and SEQ ID NO. 2, and
c. a primer that shares at least 90% sequence identity with one of the primers with SEQ ID NO. 1 and SEQ ID NO. 2 or a reverse complement thereof.

In several embodiments of the invention, the amplification step is performed using a primer pair selected from one of the groups consisting of:
a. SEQ ID NO. 1 and SEQ ID NO. 2,
b. a reverse complement of SEQ ID NO. 1 and SEQ ID NO. 2, and
c. a primer that shares at least 90% sequence identity with one of the primers with SEQ ID NO. 1 and SEQ ID NO. 2 or a reverse complement thereof.

In a preferred embodiment, the amplification is performed using a primer pair with a sequence according to SEQ ID NO. 1 and SEQ ID NO. 2.

In several embodiments of the invention, said sample originates from one of the following sample and/or tissue types comprising whole blood, blood fractions, plasma, serum, oral specimen, saliva, sputum, urine, human biopic tissue, clothing samples containing biological material, vaginal swabs, sperm, skin or wound swabs or other samples containing biological material or other parts of the human body upon availability for isolation of a genome.

Preferably, said sample comprises male and female genomic DNA.

In one embodiment of the invention, the at least two regions on the at least one locus within the Y-chromosome are not overlapping.

In another embodiment of the invention, the at least two regions on the at least one locus within the Y-chromosome are overlapping. Preferably, the at least two overlapping regions on the at least one locus within the Y-chromosome are amplified using at least one common primer.

In several embodiments of the invention, the at least one locus within the Y-chromosome is a single copy and/or a multicopy locus or at least one single copy locus and at least one multicopy locus (MCL-Y) within the Y-chromosome.

In several embodiments of the invention, the amplification step further comprises the amplification of an internal amplification control (IC).

In one embodiment of the invention, the at least two amplified regions are localized on at least two loci within the Y-chromosome.

In several embodiments of the invention, the at least two loci within the Y-chromosome are single copy and/or multicopy loci or at least one single copy locus and at least one multicopy locus (MCL-Y) within the Y-chromosome.

In one embodiment of the invention, the at least one locus within the Y-chromosome is a multicopy locus (MCL-Y), wherein said locus shares at least 85% sequence identity to a sequence according to SEQ ID NO. 3 over a stretch of at least 60 base pairs (bp) or with the reverse complement thereof, or wherein the locus is amplifiable with a primer pair according to SEQ ID NO. 1 and 2 or the reverse complement thereof.

The invention also relates to a kit for performing the method according to the invention, wherein said kit comprises at least one primer selected from the group of SEQ ID NO 1 and SEQ ID NO 2.

Digital polymerase chain reaction (dPCR; also abbreviated as e.g. digital PCR, DigitalPCR, ddPCR or dePCR) is a refinement of conventional polymerase chain reaction methods such as qPCR. Digital polymerase chain reaction (dPCR) enables the absolute quantification of target nucleic acids present in a sample and alleviates the shortcomings of qPCR. Unlike qPCR, dPCR does not rely on calibration curves for sample quantification. Hence, it avoids the pitfalls associated with variations in reaction efficiencies. dPCR is a method of absolute nucleic acid quantification that hinges on the detection of end-point fluorescent signals and the enumeration of binomial events, i.e. absence or presence of fluorescence in a partition. In dPCR, the sample is first partitioned into many independent PCR sub-reactions such that each partition contains either a few, one or no target sequences. Such partitions or microreactors can be arranged for example, but not limited to, by small water-in-oil droplets or by microfluidic nanoplates.

After PCR, the fraction of amplification-positive partitions is used to quantify the concentration of the target sequence with a statistically defined accuracy using Poisson's statistics, wherein partitions with either 0, 1, or more target sequences are each needed for the calculations. Interestingly, sample partitioning efficiently concentrates the target sequences within the isolated microreactors. This concentration effect reduces template competition and thus enables the detection of rare mutations in a background of wild-type sequences (Quan et al., 2018, MDPI, “dPCR: A Technology Review”).

dPCR may also allow for a higher tolerance to inhibitors present in a sample, because there is no need to have an amplification efficiency per cycle of almost 100%, as required for qPCR. Instead, it is sufficient if at the end of the amplification reaction either a signal or no signal is detectable. PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts.

Instead of performing one reaction per well, dPCR involves partitioning the PCR solution into at least a few hundred, but in most cases several thousand or tens of thousands or more of nano-liter sized partitions, where a separate PCR reaction takes place in each one. A dPCR solution is made similarly to a quantitative assay either using fluorescence-quencher probes or intercalating dyes, and a PCR master mix, which contains DNA polymerase, dNTPs, MgCl2, and reaction buffers at optimal concentrations.

Several different methods can be used for sample partitioning, including microwell plates, microfluidic nanoplates, capillaries, oil emulsion, and arrays of miniaturized chambers with nucleic acid binding surfaces.

After multiple PCR amplification cycles, the samples are checked for fluorescence with a binary readout of “0” (absence) or “1” (presence). The fraction of fluorescing partitions is recorded. The partitioning of the sample allows one to estimate the number of different molecules by assuming that the molecule population follows the Poisson distribution, thus accounting for the possibility of multiple target molecules inhabiting a single partition. Using Poisson's law of small numbers, the distribution of target molecule within the sample can be accurately approximated allowing for a quantification of the target strand in the PCR product.

In contrast to a qPCR reaction, a dPCR reaction is an endpoint PCR reaction. dPCR uses the number of fluorescence-positive partitions over the total to back-calculate the target concentration. In contrast to qPCR, calibration curves are not needed for sample quantification in dPCR. All in all, compared to qPCR, dPCR provides a more robust quantification, is less prone to inhibitors and is independent of a quantification standard.

The benefits of dPCR include increased precision through massive sample partitioning, which ensures reliable measurements in the desired DNA sequence due to reproducibility. Error rates are larger when detecting small-fold change differences with qPCR, while error rates are smaller with dPCR due to the smaller-fold change differences that can be detected in DNA sequence. Also, dPCR is highly quantitative as it does not rely on relative fluorescence of the solution to determine the amount of amplified target DNA.

The inventors have now astonishingly found that applying dPCR to a previously published method published in WO2018054783A1 results in entirely unexpectedly good results. The new method has an up to about 4× higher precision than the known method.

Maternal blood stream contains low amounts of cell-free fetal DNA (cffDNA) which is freely circulating. Analysis of cffDNA provides a method of non-invasive prenatal diagnosis, testing and can be used e.g. for early determination of fetal sex. The present method also enables the detection and analysis of male cell-free fetal DNA and aids in identification of fetal sex. Furthermore, the present method significantly decreases the risk of false sex determination of the embryo since contaminating male genomic DNA, e.g. introduced into the sample from the environment, can be assessed by the Degradation Index generated by the small and large PCR system for the male targets used in the invention.

### DESCRIPTION OF THE INVENTION

In case of forensic samples from sexual assaults the female DNA usually exceeds the amount of male DNA. To choose the proper method for genetic analysis it is advisable to test for male DNA present in the sample, which was collected from a crime scene and to quantify the amount of male DNA and preferably to assess in parallel the degradation status of the male DNA, in order to know how much of this DNA should be used in the genetic analysis e.g. STR reaction. The typical STR kit detects genetic length polymorphisms on different autosomal chromosomes, but in some cases, such as with sexual assault samples, the analysis of length polymorphisms exclusively on the Y-chromosome could be advantageous, because the female DNA does not contain these length polymorphisms.

The inventors have found that multicopy loci on the Y-chromosome are superior to single copy loci when used for detection and/or quantification of nucleic acids, because the sensitivity of the reaction can be enhanced. Surprisingly, the present invention now shows for example in FIG. 1 that the present method, which uses dPCR for amplification instead of qPCR, is more precise (as indicated by the lower % CV value) than previous state-of-the-art methods. This is an important aspect of the present invention due to its relevancy in the field of forensic science.

In case the locus is a multicopy locus on the Y-chromosome, it may be found many times on the Y-chromosome but not on the other chromosomes.

In case the locus is a single copy locus on the Y-chromosome, it may be present only once on the Y-chromosome but not on the other chromosomes.

The invention relates to a digital amplification method for detecting and quantifying DNA and for determining degradation and/or integrity of the DNA, in particular male DNA in a sample, wherein the method comprises the step of digital amplification of at least two regions on at least one locus within the Y-chromosome.

The invention further relates to a method for i) detecting DNA, ii) quantifying DNA, and iii) determining the integrity and/or degradation status of DNA, preferably male DNA, in a sample comprising said male DNA, wherein the method comprises the steps of:
a) amplifying at least two regions on at least one locus within the Y-chromosome, wherein the amplification is done by digital PCR,
b) detecting the at least two amplification products through the use of at least two probes,
c) quantifying the amount of the at least two amplification products, and
d) determining the integrity and/or degradation status of the DNA of the sample by calculating a degradation index,
   wherein at least one of the at least two probes binds to one of the at least two amplification products and at least one other probe of the at least two probes binds to another one of the at least two amplification products, and wherein at least one of the at least two amplification products is longer than another one of the at least two amplification products.

In one embodiment of the invention, the at least two regions being amplified are localized on at least one locus within the Y-chromosome, but are either not overlapping with each other or at least two of the two or more regions are not overlapping. The at least one locus can be a single copy or multicopy locus (MCL-Y) within the Y-chromosome.

In another embodiment of the invention, the at least two regions being amplified are localized on at least two loci within the Y-chromosome. The loci can be single copy and/or multicopy loci (MCL-Y) within the Y-chromosome.

In yet another embodiment of the invention, the at least two regions being amplified are localized on at least one locus within the Y-chromosome and are overlapping with each other. The at least one locus can be a single copy or multicopy locus (MCL-Y) within the Y-chromosome.

In particular, the inventors have astonishingly found that sequence identified in SEQ ID NO. 3 and/or sequences that share sequence similarity, i.e. SEQ ID NO. 4 with it may be found many times on the Y-chromosome. In particular, said sequence SEQ ID NO. 3 or sequences very similar thereto are present nine times on the human Y-chromosome. This finding provides a valuable advantage of the present method.

In one embodiment of the invention, the amplification step is performed using at least one primer selected from one of the groups consisting of:
a. SEQ ID NO. 1 and SEQ ID NO. 2,
b. a reverse complement of SEQ ID NO. 1 and SEQ ID NO. 2, and
c. a primer that shares at least 90% sequence identity with one of the primers with

SEQ ID NO. 1 and SEQ ID NO. 2 or a reverse complement thereof.

In several embodiments of the invention, the amplification step is performed using a primer pair selected from one of the groups consisting of:
a. SEQ ID NO. 1 and SEQ ID NO. 2,
b. a reverse complement of SEQ ID NO. 1 and SEQ ID NO. 2, and
c. a primer that shares at least 90% sequence identity with one of the primers with SEQ ID NO. 1 and SEQ ID NO. 2 or a reverse complement thereof.

In a preferred embodiment, the amplification is performed using a primer pair with a sequence according to SEQ ID NO. 1 and SEQ ID NO. 2.

In several embodiments of the invention, said sample originates from one of the following tissue types comprising whole blood, blood fractions, oral specimen, urine, human biopic tissue or other parts of the human body upon availability for isolation of a genome.

Preferably, said sample comprises male and female genomic DNA.

In one embodiment of the invention, the at least two regions on the at least one locus within the Y-chromosome are not overlapping.

In another embodiment of the invention, the at least two regions on the at least one locus within the Y-chromosome are overlapping. Preferably, the at least two overlapping regions on the at least one locus within the Y-chromosome are amplified using at least one common primer.

In several embodiments of the invention, the at least one locus within the Y-chromosome is a single copy and/or a multicopy locus or at least one single copy locus and at least one multicopy locus (MCL-Y) within the Y-chromosome.

In several embodiments of the invention, the amplification step further comprises the amplification of an internal amplification control (IC). In one embodiment, the internal amplification control is an artificial nucleic acid template. In an alternative embodiment, the internal amplification control comprises a sequence of an organism, which is different than the origin or suspected origin of the sample nucleic acid to be analyzed. In a preferred embodiment, the internal amplification control comprises a sequence with a very low identity to sequences in the sample. In a preferred embodiment, the internal amplification control has been generated using a random algorithm. In a more preferred embodiment the internal amplification control is similar to that of SEQ ID NO 48. In a preferred embodiment, the internal amplification control has a length between 70 and 2000 nucleotides. In a preferred embodiment, the internal amplification control has a length between 150 and 1500 nucleotides. In a more preferred embodiment the internal amplification control has a length between 150 and 1000 nucleotides. In an even more preferred embodiment the internal amplification control has a length between 200 and 500 nucleotides. More preferably, the internal amplification control has a length between 50 and 2000 nucleotides. In a preferred embodiment, the internal amplification control has a length between 50 and 1500 nucleotides. In a more preferred embodiment the internal amplification control has a length between 50 and 1000 nucleotides. In an even more preferred embodiment the internal amplification control has a length between 50 and 500 nucleotides. Analysis of the target amplification products and the internal control fragments allows for the differential identification of the presence of inhibitors or degradation of the sample. A more detailed description of structural and functional features of the internal amplification control is provided in WO2018050835A1, which is hereby incorporated by reference.

In one embodiment of the invention, the at least two amplified regions are localized on at least two loci within the Y-chromosome.

In several embodiments of the invention, the at least two loci within the Y-chromosome are single copy and/or multicopy loci or at least one single copy locus and at least one multicopy locus (MCL-Y) within the Y-chromosome.

In one embodiment of the invention, the at least one locus within the Y-chromosome is a multicopy locus (MCL-Y), wherein said locus shares at least 85% sequence identity to a sequence according to SEQ ID NO. 3 over a stretch of at least 60 base pairs (bp) or with the reverse complement thereof, or wherein the locus is amplifiable with a primer pair according to SEQ ID NO. 1 and 2 or the reverse complement thereof.

The invention also relates to a kit for performing the method according to the invention, wherein said kit comprises at least one primer selected from the group of SEQ ID NO 1, SEQ ID NO 2, SEQ ID NO 13, SEQ ID NO 14 and/or SEQ ID NO 15 and/or complements thereof and optionally at least one probe selected from the group of SEQ ID NO 12 and/or SEQ ID NO 16 and/or complements thereof.

In the context of the present invention, the term “amplifiable” refers to the property of a region of being amplified by a dPCR amplification method. A person skilled in the art knows that the achievement of the amplification reaction depends on the experimental condition used.

The invention relates to a method, wherein the digital amplification step is performed using at least one primer selected from one of the groups consisting of (i) SEQ ID NO. 1 and SEQ ID NO. 2, (ii) a reverse complement of SEQ ID NO. 1 and SEQ ID NO. 2 and (iii) a primer that shares at least 90% sequence identity with one of the primers with SEQ ID NO. 1 and SEQ ID NO. 2 or a reverse complement thereof.

| | |
|---|---|
| SEQ ID NO. 1 | 5′GAAAGGCCTCATCAGGGCTCAG 3′ |
| Downstream | |
| primer 2 81 bp | |
| fragment | |
| SEQ ID NO. 2 | 5′TCCTCACTGGGAAACATGAGGAATGAC 3′ |
| Upstream | |
| primer 1 | |

The invention relates to a digital amplification method, wherein the digital amplification step is performed using a primer pair selected from one of the groups consisting of (i) SEQ ID NO. 1 and SEQ ID NO. 2, (ii) a reverse complement of SEQ ID NO. 1 and SEQ ID NO. 2 and (iii) a primer that shares at least 90% sequence identity with one of the primers with SEQ ID NO. 1 and SEQ ID NO. 2 or a reverse complement thereof.

The sequences distributed throughout the genome are not all identical. It is important that the selected primers bind also to the nearly identical sequences. Thus, ideally the region shares at least 60%, 70%, 80%, 90% or even 95% or 98% sequence identity to a sequence according to SEQ ID NO. 3 over a stretch of 60 bp.

The region may also be chosen from any of SEQ ID NO. 3 to 11. Thus, if SEQ ID NO. 3 is claimed herein the same applies to 4 to 11.

| | |
|---|---|
| SEQ ID NO. 3 | 5′GAAAGGCCTCATCAGGGCTCAGaaaggtgacccaagca |
| (chrY: 9529589- | gctgggaacacacgggGTCATTCCTCATGTTTCCCAGTGA |
| 9529669 81bp) | GGA 3′ |
| SEQ ID NO. 4 | 5′GAAAGGCCTCATCAGGGCTCAGaaaggtgacccaagca |
| (chrY: 9509276- | gctgggaacacacgggGTCATTCCTCATGTTTCCCAGTGA |
| 9509356 81bp) | GGA 3′ |
| SEQ ID NO. 5 | 5′GAAAGGCCTCATCAGGGCTCAGaaaggtgacccaagca |
| (chrY: 9488994- | gctgggaacacatgggGTCATTCCTCATGTTTCCCAGTGA |
| 9489074 81bp) | GGA 3′ |
| SEQ ID NO. 6 | 5′GAAAGGCCTCATCAGGGCTCAGaaaggtgacccaagca |
| (chrY: 9468664- | gctgggaacacacgggGTCATTCCTCATGTTTCCCAGTGA |
| 9468744 81bp) | GGA 3′ |
| SEQ ID NO. 7 | 5′GAAAGGCCTCATCAGGGCTCAGaaaggtgacccaagca |
| (chrY: 9400130- | gctgggaacacacgggGTCATTCCTCATGTTTCCCAGTGA |
| 9400210 81bp) | GGA 3′ |
| SEQ ID NO. 8 | 5′GAAAGGCCTCATCAGGGCTCAGaaaggtgacccaagca |
| (chrY: 9379784- | gctgggaacacacgggGTCATTCCTCATGTTTCCCAGTGA |
| 9379864 81bp) | GGA 3′ |
| SEQ ID NO. 9 | 5′GAAAGGCCTCATCAGGGCTCAGaaaggtgacccaagca |
| (chrY: 9359506- | gctgggaacacacgggGTCATTCCTCATGTTTCCCAGTGA |
| 9359586 81bp) | GGA 3′ |
| SEQ ID NO. 10 | 5′GAAAGGCCTCATCAGGGCTCAGaaaggtgacccaagca |
| (chrY: 9339191- | gctgggaacacatgggGTCATTCCTCATGTTTCCCAGTGA |
| 9339271 81bp) | GGA 3′ |
| SEQ ID NO. 11 | 5′GAAAGGCCTCATCAGGGCTCAGaaaggtgacccaagca |
| (chrY: 6247926- | gctgggaacacacgggGTCATTCCTCATGTTTCCCAGTGA |
| 6248006 81bp) | GGA 3′ |
| SEQ ID NO. 12 | 5′ ggtgacccaagcagctgggaacaca 3′ |
| Probe for 81 bp | |
| fragment | |
| SEQ ID NO. 13 | 5′ CATGAACGTCCTGGATTCTGTCACTC 3′ |
| Downstream primer | |
| no. 3.2 | |
| SEQ ID NO. 14 | 5′ TCACTCTCTGTCTTCCTCTCAAGGAATTTCTAC 3′ |
| Downstream primer | |
| no. 3.1 | |
| SEQ ID NO. 15 | 5′ GCCATGAACGTCCTGGATTCTGTCAC 3′ |
| Downstream primer | |
| no. 3.3 | |
| SEQ ID NO. 16 | 5′ CAGGCTCCCTGAATAGGCAGGTGTG 3′ |
| Probe 2 for larger | |
| fragment | |
| SEQ ID NO: 17 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13- | cctgaagagggaacagagacgtcaggtgagccgttagttg |
| Region: chrY: | gcactggagctgtttgatgcccagtataagggggttgaca |
| 9529589+9529947) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 18 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9509276+9509634) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 19 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9488994+9489352) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 20 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcgcagtataagggggttgaca |
| 9468664+9469020) | cacctgcctattcagggagcctgggtgctcatttcagaaa |
| | tgtagaaactgaggctcctttcgtacatgtagaaattcct |
| | tgagaggaagacaGAGTGACAGAATCCAGGACGTTCATG |
| | 3′ |
| SEQ ID NO. 21 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9400130+9400488) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 22 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9379784+9380142) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 23 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9359506+9359864 | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 24 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 9339191+9339549) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 25 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 13 | cctgaagagggaacagagacgtcaggtgagccgttagttg |
| Region: chrY: | gcactggagctgtttgatgcccagtataagggggttgaca |
| 6247926+6248284) | cacctgcctattcagggagcctgggtgctcatttcagaaa |
| | tgtagaaattgaggctcctttcgtacatgtagaaattcct |
| | tgagaggaagacagaGAGTGACAGAATCCAGGACGTTCAT |
| | G 3′ |
| SEQ ID NO. 26 | 5′TCCTCACTGGGAAACATGAGGAATGACcccatgtgttc |
| (Upstream primer | ccagctgcttgggtcaccattctgagtcctgatgaggcct |
| SEQ ID NO. 2 and | ttcccgatggattcccctgacagatcctatgtaaggacct |
| Downstream primer | gtggtgcaatcccctgcaatcctacaagaggatgaagcca |
| SEQ ID NO. 14 | cctgaagagggaacagagatttcaggtgagctgttcagtt |
| Region: chrY: | ggaactgaagctttttgatccccaggataaggaggttgac |
| 9549757+9550096) | acacctgcctattcagggagcctggaggctcatttcagaa |
| | atgtagaaattgagcctcctttcatacatGTAGAAATTCC |
| | TTGAGAGGAAGACAGAGtGTGA 3′ |
| SEQ ID NO. 27 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9529589+9529927 | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 28 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9509276+9509614) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 29 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9488994+9489332) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 30 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcgcagtataagggggttgaca |
| 9468664+9469002) | cacctgcctattcagggagcctgggtgctcatttcagaaa |
| | tgtagaaactgaggctcctttcgtacatGTAGAAATTCCT |
| | TGAGAGGAAGACAGAGtGacA 3′ |
| SEQ ID NO. 31 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9400130+9400468) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 32 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9379784+9380122) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 33 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9359506+9359844) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 34 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 9339191+9339529) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 35 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 14 | cctgaagagggaacagagacgtcaggtgagccgttagttg |
| Region: chrY: | gcactggagctgtttgatgcccagtataagggggttgaca |
| 6247926+6248264) | cacctgcctattcagggagcctgggtgctcatttcagaaa |
| | tgtagaaattgaggctcctttcgtacatGTAGAAATTCCT |
| | TGAGAGGAAGACAGAGAGTGA 3′ |
| SEQ ID NO. 36 | 5′TCCTCACTGGGAAACATGAGGAATGACcccatgtgttc |
| (Upstream primer | ccagctgcttgggtcaccattctgagtcctgatgaggcct |
| SEQ ID NO. 2 and | ttcccgatggattcccctgacagatcctatgtaaggacct |
| Downstream primer | gtggtgcaatcccctgcaatcctacaagaggatgaagcca |
| SEQ ID NO. 15 | cctgaagagggaacagagatttcaggtgagctgttcagtt |
| Region: chrY: | ggaactgaagctttttgatccccaggataaggaggttgac |
| 9549757+9550118) | acacctgcctattcagggagcctggaggctcatttcagaa |
| | atgtagaaattgagcctcctttcatacatgtagaaattcc |
| | ttgagaggaagacagagtGTGACAGAATCCAGGACaTTCA |
| | TGGC 3′ |
| SEQ ID NO. 37 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9529589+9529949) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 38 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9509276+9509636) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 39 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9488994+9489354) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 40 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcgcagtataagggggttgaca |
| 9468664+9469022) | cacctgcctattcagggagcctgggtgctcatttcagaaa |
| | tgtagaaactgaggctcctttcgtacatgtagaaattcct |
| | tgagaggaagacagaGTGACAGAATCCAGGACGTTCATGG |
| | C 3′ |
| SEQ ID NO. 41 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9400130+9400490) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 42 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9379784+9380144) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 43 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9359506+9359866) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 44 | 5′TCCTCACTGGGAAACATGAGGAATGACcccatgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 9339191+9339551) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 45 | 5′TCCTCACTGGGAAACATGAGGAATGACcccgtgtgttc |
| (Upstream primer | ccagctgcttgggtcacctttctgagccctgatgaggcct |
| SEQ ID NO. 2 and | ttcccgattgagtcccctgacagatcctatgtaaggacct |
| Downstream primer | gtggcgcaatcccctgcaatactacaagaggatgaagcca |
| SEQ ID NO. 15 | cctgaagagggaacagagacgtcaggtgagccgttagttg |
| Region: chrY: | gcactggagctgtttgatgcccagtataagggggttgaca |
| 6247926+6248286) | cacctgcctattcagggagcctgggtgctcatttcagaaa |
| | tgtagaaattgaggctcctttcgtacatgtagaaattcct |
| | tgagaggaagacagagaGTGACAGAATCCAGGACGTTCAT |
| | GGC 3′ |
| SEQ ID NO. 46 | GTAGCTCCATAATCAGAAGATGCG |
| (IC primer 1) | |
| SEQ ID NO. 47 | ATTCGCTCTACTCCAACCACATAATAG |
| (IC primer 2) | |
| SEQ ID NO. 48 | GTAGCTCCATAATCAGAAGATGCGctaccgtacacaacggc |
| (IC template) | ggtttaaactttgtagacctcacgcggctgctcaggtcttg |
| | cgcatcccatacttcacatggacggaaagctgtgggtttgt |
| | atcgataagccgtaggttcctaggatcgagtcacccaaaca |
| | acttcgagagctcttctcaccaaccctttgaactccgaaat |
| | gccgtgaacacatccccttagttggcattggtctactaacc |
| | gagtatcgtacaaagcgagacaggtctggcccctactgctt |
| | ggatgcgaactaacagtgttcctatatccgtttatactgtg |
| | gtcgatttcgaaccgatatctcaaagagagcgccggcagag |
| | ccttagtatcggaactggcgagtgtgtttgggtgaggaCTA |
| | TTATGTGGTTGGAGTAGAGCGAAT |
| SEQ ID NO. 49 | TCCGATACTAAGGCTCTGCCGGCGCT |
| (IC probe) | |
| SEQ ID NO 50 | CCTGAGGATGCCACAGTGAGACAC |
| Human Primer 1 | |
| SEQ ID NO 51 | ACTTCTGTCTACTGTCGGACTCTACAGG |
| Human Primer 2 | |
| SEQ ID NO 52 | GTGGCCCATTTAGGCCCAACTC |
| Human Primer 3.1 | |
| (200 bp) | |
| SEQ ID NO 53 | GGCCCAGCTTTGACTTGAGAACAG |
| Human Primer 3.2 | |
| (353 bp) | |
| SEQ ID NO 54 | GCTGGGTCTACAGGCACAACTG |
| Human Primer 3.3 | |
| (496 bp) | |
| SEQ ID NO 55 | TCTGGAGGGTCCACTGTGAGGCAG |
| Human Probe 1 | |
| SEQ ID NO 56 | CCATTTCCAGGCCTAGTGCCTGCC |
| Human Probe 2 | |
| SEQ ID NO 57 | TGCCTGTTGAGGCTGCTGGCAGGC |
| Human Probe 3 | |

The inventors have astonishingly developed a very versatile dPCR system for degradation analysis.

The degradation status/integrity of male DNA can be assessed by using for example, at least two differently sized genomic regions in a dPCR in one vessel. Preferably, the amplified targets either have comparable copy numbers, or the lack of it is accounted for in subsequent calculations. In case of degraded male DNA, the mean length of the male DNA fragments in the sample will decrease. The Degradation Index is calculated by dividing the quantity of a smaller amplicon by the quantity of a larger amplicon. The more fragmented the template DNA the higher the degradation index because of a significant loss of possible template molecules for the larger amplicon. As an example, having a mean DNA size of 150 bp for a plurality of DNA molecules, wherein the size of the plurality of DNA molecules follows a Gaussian distribution, implies that only few molecules with a size of 353 bp and even less molecules having a size of 496 bp are present. Therefore, the degradation index allows a conclusion to the state of degradation of the unknown DNA in the customers sample. Hence, in one aspect of the present digital amplification method, the status of DNA integrity and/or degradation is expressed by the ratio of the quantification of the at least two overlapping and/or non-overlapping regions within the at least one locus.

Therefore, in one aspect of the present invention, the status of DNA integrity and/or degradation is expressed by the ratio of the quantification of the at least two overlapping and/or non-overlapping regions within the at least one locus.

Here, the smaller fragment is combined with different larger fragments, depending on the extent of degradation of the nucleic acid.

The larger fragment may be a:
(i) 359 bp or 357 bp fragment (primers SEQ ID NO. 2 and SEQ ID NO. 13),
(ii) a 340 bp fragment or a 339 bp fragment (e.g. primers SEQ ID NO. 2 and SEQ ID NO. 14), or a
(iii) 359 bp fragment, 361 bp fragment or a 362 bp fragment (SEQ ID NO. 2 and SEQ ID NO. 15)

Uniquely the system is set-up so that one of the primers in the primer pairs is common to the two or more fragments, being amplified. Thus, the two fragments small and large may have a common up-stream or down-stream primer. Here, it is preferred that the upstream primer (SEQ ID NO. 2) is common to all amplifications.

The determination of percent identity between two sequences is accomplished using the mathematical algorithm of Karlin and Altschul (Proc. Natl. Acad. Sci. USA (1993) 90:5873-5877). Such an algorithm is the basis of the BLASTN and BLASTP programs of Altschul et al. (J. Mol. Biol. (1990) 215:403-410). BLAST nucleotide searches are performed with the BLASTN program, score=100, word length=12, to obtain nucleotide sequences homologous SEQ ID NO. 1. To obtain gapped alignments for comparative purposes, Gapped BLAST is utilized as described by Altschul et al. (Nucleic Acids Res. (1997) 25:3389-3402). When utilizing BLAST and Gapped BLAST programs, the default parameters of the respective programs are used.

The forensic workflow of sexual assault samples suggests the quantification of the male DNA before the STR reaction is carried out. This is done first to help in the decision of which kind of STR Kit has to be used for the genetic analysis, and then to determine how much DNA was obtained from a sample, e.g. collected from a crime scene, and how much of this DNA should be used in a STR reaction. Different STR Kits are available, the typical STR Kit detects genetic length polymorphisms on different autosomal chromosomes, but in some cases, such as with sexual assault samples, the analysis of length polymorphisms exclusively on the Y-chromosome could be advantageous, because the female DNA doesn't have a Y chromosome.

The typical STR reaction works optimally in certain range of template DNA and the whole analysis is very labour-intensive, therefore methodologies are needed that ensure a very high success rate in the STR analysis. Therefore, it is a real advantage if the quantification kit enables the user not only to surely identify the amount of DNA present but also to assess the absence of inhibitors, which could compromise the STR reaction result, which would result in failure or loss of valuable sample material, which could be further purified in case critical inhibition is observed.

According to one embodiment of the present invention, said region on the multicopy locus within the human Y-chromosome (MCL-Y) is about 81 bp in length. As used herein, the term “about” refers to a range comprising +/−20% of the value of reference. Thus, said region on the multicopy locus can have a length ranging from 65 to 95 bp.

Preferably, the method comprises the step of digital amplification of at least two regions on at least one multicopy locus within the Y-chromosome (MCL-Y), wherein said locus shares at least 85%, 90%, 95% or 99% sequence identity to a sequence according to SEQ ID NO. 3 over a stretch of at least 60 base pairs (bp) or with the reverse complement thereof.

The present digital PCR method shows an improved precision over other commercially available methods.

According to another embodiment of the present digital application, the amplification product of at least one nucleic acid is between 20 and 200 bp in length.

Preferably, the digital amplification step is performed using at least one primer selected from one of the groups consisting of (i) SEQ ID NO. 1 and SEQ ID NO. 2, (ii) a reverse complement of SEQ ID NO. 1 and SEQ ID NO. 2 and (iii) a primer that shares at least 90% sequence identity with one of the primers with SEQ ID NO. 1 and SEQ ID NO. 2 or a reverse complement thereof. These may be combined with a second overlapping amplicon wherein the primer pairs used have a sequence according to (iv) SEQ ID NO. 2 and SEQ ID NO. 13, SEQ ID NO. 2 and SEQ ID NO. 14, (v) and SEQ ID NO. 2 and SEQ ID NO. 15; see also FIG. 2. When measuring additionally degradation, the amplicon SEQ ID NO. 1 and 2 is combined with an amplicon selected from (i) SEQ ID NO. 2 and SEQ ID NO. 13, (ii) SEQ ID NO. 2 and SEQ ID NO. 14, or (iii) SEQ ID NO. 2 and SEQ ID NO. 15.

In a preferred embodiment the digital amplification reaction comprises amplifying at least two overlapping regions using at least one common primer, or two common primers or three common primer or more.

Preferably, the amplification step is performed using a primer pair selected from one of the groups consisting of (i) SEQ ID NO. 1 and SEQ ID NO. 2, (ii) a reverse complement of SEQ ID NO. 1 and SEQ ID NO. 2, and (iii) a primer that shares at least 90% sequence identity with one of the primers with SEQ ID NO. 1 and SEQ ID NO. 2 or a reverse complement thereof optionally combined with one or more of the pairs selected from (i) SEQ ID NO. 2 and SEQ ID NO. 13, (ii) SEQ ID NO. 2 and SEQ ID NO. 14, or (iii) SEQ ID NO. 2 and SEQ ID NO. 15.

Ideally, the digital amplification is performed using a primer pair with a sequence according to SEQ ID NO. 1 and SEQ ID NO. 2.

In some embodiments, an amplification product may be at least 10%, 15%, 20% 25%, 30%, 35%, 40%, 45%, or 50% longer than another amplification product. If three amplification products are present, the third one may be at least 10%, 15%, 20% 25%, 30%, 35%, 40%, 45%, or 50% longer than the second amplification product. If four amplification products are present, the fourth one may be at least 10%, 15%, 20% 25%, 30%, 35%, 40%, 45%, or 50% longer than the third amplification product, and so on. Hence, preferably, the amplification products are each of different sizes. Alternatively, only one of the at least two amplification products has a different size, which may be at least 10%, 15%, 20% 25%, 30%, 35%, 40%, 45%, or 50% longer than at least one other amplification product.

Preferably, the sample originates from one of the following sample and/or tissue types comprising whole blood, blood fractions, plasma, serum, oral specimen, saliva, sputum, urine, human biopic tissue, clothing samples containing biological material, vaginal swabs, sperm, skin or wound swabs or other samples containing biological material or other parts of the human body upon availability for isolation of a genome.

The present method also enables the detection and analysis of the degradation status of male DNA in non-degraded or also degraded female DNA.

Preferably, the digital amplification method is a digital polymerase chain reaction (PCR).

According to another embodiment, the sample subjected to the present method originates from one of the following specimens comprising whole blood, blood fractions, oral fluids, body fluids, human bioptic tissue or other parts of the human body upon availability for isolation of a genome. As used herein the terms “oral fluids” and “body fluids” refers to fluids that are excreted or secreted from the buccal cavity and from the body, respectively, from which a genome can be isolated. As a non-limiting example, oral and body fluids may comprise saliva, sputum, swab, urine.

In a preferred embodiment, the DNA or RNA analyzed is fragmented. In another embodiment, the DNA or RNA analyzed is in a composition with inhibitors.

As reported above, a typical forensic sample comprise a mixture of male and female DNA wherein the amount of female DNA exceeds the amount of male DNA by several orders of magnitude. Thus, according to another embodiment, the sample comprises one or more additional nucleic acids originating from a different genome. As used herein, the term “different genome” refers to genome isolated from a different subject, generally identified as female DNA.

According to another embodiment of the present invention, the digital amplification method is a digital polymerase chain reaction (PCR).

The amplification reaction according to the present method may be either a non-isothermal method or an isothermal method.

The amplification methods will comprise buffers, dNTPs or NTPs in addition to the enzymes required.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescent label, a radioactive label, a biotin label.

dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine. Furthermore, ddNTPs of the above-described molecules are encompassed in the present invention.

As used herein, the term “NTP” refers to ribonucleoside triphosphates. Non-limiting examples of such NTPs are ATP, GTP, CTP, TTP, UTP, which may also be present in the form of labelled derivatives, for instance comprising a fluorescent label, a radioactive label, a biotin label.

According to one embodiment of the present invention, the amplification reaction comprises, (a) a buffer at a pH of between 7.8 and 9 or between 8 and 8.8 (at 20° C.) and/or, (b) potassium salt selected from the group of, potassium chloride and potassium sulphate and/or, (c) an ammonium salt, preferably ammonium chloride or ammonium sulphate and/or, (d) magnesium chloride and/or, (e) a polymerase, preferably a hot-start polymerase.

Suitable buffers may be Tris-HCl-, HEPES-, TEA- and/or MOPS-buffers but any other suitable buffer may be used.

According to another embodiment of the present invention, the amplification reaction comprises, (a) Tris-HCl at a pH of between 8 and 8.8 (at 20° C.) and/or, (b) potassium salt selected from the group of, potassium chloride and potassium sulphate and/or, (c) an ammonium salt, preferably ammonium chloride or ammonium sulphate and/or, (d) magnesium chloride and/or, (e) a hot-start polymerase.

Preferably, the concentration of Tris-HCl is in the range from 10 to 100 mM, most preferably in the range from 20 to 70 mM, the concentration of K⁺ is in the range from 1-25 mM, most preferred in the range from 2.5 to 20 mM, the concentration of NH₄⁺ in range from 1 to 40 mM, most preferred in the range from 2.5 to 30 mM, and a concentration of Mg²⁺ of 0.5 mM to 8 mM in excess to the concentration of the four dNTP's, most preferred a concentration of Mg²⁺ of 0.7 mM to 5 mM in excess to the concentration of the four dNTP's, a hot-start polymerase, preferentially a hot-start polymerase allowing a hot-start time of less than 5 min, most preferred below 2 min.

A second aspect of the present invention relates to a primer or primer pair for digitally amplifying at least one region within a multicopy locus within the human Y-chromosome (MLC-Y) selected from the group consisting of: 5′ GAAAGGCCTCATCAGGGCTCAG 3′ (SEQ ID NO 1) and 5′ TCCTCACTGGGAAACATGAGGAATGAC 3′ (SEQ ID NO 2).

According to an embodiment of the second aspect, at least one primer hybridizes under stringent conditions to a region of the Y-chromosome represented by a region on a multicopy locus according to SEQ ID NO. 3 to SEQ ID NO. 11.

The primers and probes according to the invention may be in a kit, the kit further optionally including the primers and probe for the internal amplification control. Hence, the invention also relates to a kit for detecting and quantifying DNA and for determining degradation and/or integrity of the DNA, in particular male DNA in a sample, wherein the method comprises the step of digital amplification of at least two regions within at least one locus within the Y-chromosome.

The at least one locus within the Y-chromosome may be a single copy and/or a multicopy locus (MCL-Y).

Said kit comprises at least one primer, that under stringent conditions, binds a sequence that shares at least 80% sequence identity to a sequence according to SEQ ID NO. 3 to 11 over a stretch of 80 bp, wherein in a digital amplification reaction, at least one region within at least one locus within the Y-chromosome is amplified. The locus is a single copy and/or a multicopy locus within the human Y-chromosome. The at least one primer can bind at least SEQ ID NO. 3, 4, 5, 6, 7, 8, 9, 10 and/or SEQ ID NO. 11 or all of the above.

In particular, the invention relates to a kit comprising at least one primer selected from the group of SEQ ID NO 1, SEQ ID NO 2, SEQ ID NO 13, SEQ ID NO 14 and/or SEQ ID NO 15 and/or complements thereof and optionally at least one probe selected from the group of SEQ ID NO 12 and/or SEQ ID NO 16 and/or complements thereof, for detecting and quantifying DNA and for determining degradation and/or integrity of the DNA, in particular male DNA, in a sample, in a digital amplification reaction of at least one region within at least one locus within the Y-chromosome. The kit optionally further comprises one or more reagents needed to perform the methods of the invention, e.g. one or more intercalating dyes, a DNA polymerase, dNTPs, MgCl2, and a reaction buffer.

The invention further relates to a kit for at least a triplex dPCR reaction comprising primers and/or probes for detecting and quantifying DNA and for determining degradation and/or integrity of the DNA, in particular male DNA, in a sample in a digital amplification reaction of at least one region within at least one locus within the Y-chromosome, said kit further comprising an internal amplification control (IC) and primers and/or probes for detecting and quantifying said internal amplification control.

In particular, the invention further relates to a kit for at least a triplex dPCR reaction comprising at least one primer selected from the group of SEQ ID NO 1, SEQ ID NO 2, SEQ ID NO 13, SEQ ID NO 14 and/or SEQ ID NO 15 and/or complements thereof and optionally at least one probe selected from the group of SEQ ID NO 12 and/or SEQ ID NO 16 and/or complements thereof for detecting and quantifying DNA and for determining degradation and/or integrity of the DNA, in particular male DNA, in a sample in a digital amplification reaction of at least one region within at least one locus within the Y-chromosome, said kit further comprising an internal amplification control (IC) according to SEQ ID NO 48 and primers and/or probes according to SEQ ID NO 46, SEQ ID NO 47 and/or SEQ ID NO 49 for detecting and quantifying said internal amplification control.

In another embodiment, the invention relates to a kit for at least a pentaplex (5-plex) dPCR reaction comprising primers and/or probes for detecting and quantifying DNA and for determining degradation and/or integrity of the DNA, in particular male DNA, in a sample in a digital amplification reaction of at least one region within at least one locus within the Y-chromosome, said kit further comprising an internal amplification control (IC) and primers and/or probes for detecting and quantifying said internal amplification control and primers and/or probes for detecting and quantifying DNA and for determining degradation and/or integrity of DNA, in particular human or female DNA.

In particular, the invention further relates to a kit for at least a pentaplex (5-plex) dPCR reaction comprising at least one primer selected from the group of SEQ ID NO 1, SEQ ID NO 2, SEQ ID NO 13, SEQ ID NO 14 and/or SEQ ID NO 15 and/or complements thereof and optionally at least one probe selected from the group of SEQ ID NO 12 and/or SEQ ID NO 16 and/or complements thereof for detecting and quantifying DNA and for determining degradation and/or integrity of the DNA, in particular male DNA, in a sample in a digital amplification reaction of at least one region within at least one locus within the Y-chromosome, said kit further comprising an internal amplification control (IC) according to SEQ ID NO 48 and primers and/or probes according to SEQ ID NO 46, SEQ ID NO 47 and/or SEQ ID NO 49 for detecting and quantifying said internal amplification control and said kit further comprises at least one primer selected from the group of SEQ ID NO 50, SEQ ID NO 51, SEQ ID NO 52, SEQ ID NO 53 and/or SEQ ID NO 54 and/or complements thereof and/or at least one probe selected from the group of SEQ ID NO 55, SEQ ID NO 56 and/or SED ID NO 57 for detecting and quantifying DNA and for determining degradation and/or integrity of the DNA, in particular human or female DNA, in a sample in a digital amplification reaction.

In one embodiment of the invention, the digital amplification reaction is a multiplex dPCR, wherein at least 5 different targets (human, degradation, male, male degradation and internal amplification control (IC)) are amplified.

Astonishingly, the inventors found that the invention is highly multiplexable. Thus, they were able to run at least 5 different targets (human, degradation, male, male degradation and internal amplification control (IC)) that have been successfully amplified in parallel (Example 5 and FIG. 5). The detection of further single copy- and/or multicopy loci and/or biomarkers e.g. for use in forensics, prenatal diagnostics, human identification, may also be possible in parallel by higher multiplexing.

The invention further relates to a method for obtaining a degradation index for male DNA of at least 7 when measuring degraded DNA of 300 bp length and of at least 237 when measuring degraded male DNA of 150 bp length.

Preferably in the digital amplification method according to the invention, the assessment of the status of male DNA degradation and/or integrity of one or more nucleic acids in a sample is done in parallel with the detection of the one or more nucleic acids that are quantified. Preferably, the method addresses the status of male DNA degradation and/or integrity even in the presence of high background female DNA. In a further embodiment, the method may also be used for non-invasive early determination of fetal sex.

The invention relates also in particular to assessing the status of male DNA in the sample. This is done to assess the integrity or degradation status of the male DNA in the sample.

### EXAMPLES AND FIGURES

Example 1 and FIG. 1 shows precision data on male DNA quantification by digital PCR compared to state-of-the-art qPCR methods.

Shown are the male DNA quantification data incl. mean values, standard deviation and coefficient of variation (CV) for digital PCR and two different state of the art qPCR quantification methods for male DNA (QIAGEN Investigator Quantiplex Pro Kit and QIAGEN Investigator Quantiplex HYres Kit). The coefficient of variation (CV) is defined as the ratio of the standard deviation divided by the mean. It shows the extent of variability in relation to the mean of the population. The lower the CV value the higher the precision for quantification and predicting the degree of degradation even if only single replicates of one sample are used. Noticeably, the method according to the invention (dPCR) demonstrates much higher precision compared to qPCR, for the male target (male) in digital PCR the CV is 2.6% compared to male on qPCR where it is 5.2% for QPro and even 10.5% for QHYres. For the male degradation target the coefficient of variance is at 2.9% for digital PCR and at 8.9% for QPro on qPCR. These data clearly show a much higher precision and reliability for quantification of male DNA with the invention (na=not available).

Example 2 and FIG. 2 shows measurement of male DNA integrity by degradation index according to the invention.

Degraded male DNA with an average fragment length from 1500 bp, 1000 bp, 800 bp, 500 bp, 400 bp, 300 bp, 200 bp and 150 bp has been applied to the method of invention. Male Degradation indices (MDI) have been calculated by dividing the quantification value for male target (small target) by the quantification value of the male degradation target (larger target). The MDI value increases with higher degradation of the quantified DNA. The method of invention generates MDIs with high differences between degraded male DNA fragments of 400 bp vs 300 bp vs 200 bp vs 150 bp size.

Example 3 and FIG. 3 shows a comparison of male degradation indices between invention and state of the art methods. FIG. 3 shows male degradation indices (i.e. the ratio of the amount of short fragments vs. the amount of long fragments (male/male degradation) of the different systems tested. Noticeably, the method according to the invention (second column) obtains consistently higher indices, in particular for the smaller fragments ranging for 300 bp and 150 bp (a value of more than 237) compared to the other systems on qPCR. This indicates a much higher sensitivity/precision for the detection of degraded male DNA and better resolution of male DNA integrity.

Example 4 and FIG. 4 shows measurement of degraded male DNA in background of non-degraded female DNA.

FIG. 4 shows the measurement of degraded male DNA in background of non-degraded female DNA. Different fragmented male DNAs (each 0.04 ng/R×N) have been spiked into non-degraded female DNA (40 ng/R×N). The invention shows a significant increase for the MDI values for the fragment length from 500 bp to 200 bp, while the human degradation index is not able to detect the degraded male DNA in female DNA background and report male DNA degradation. The invention allows for a precise assessment of the degradation or integrity status of male DNA in female background DNA.

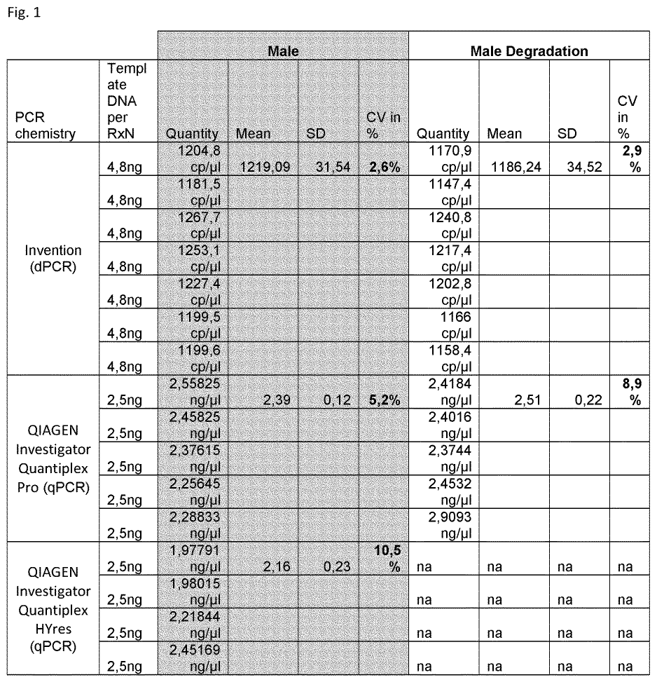

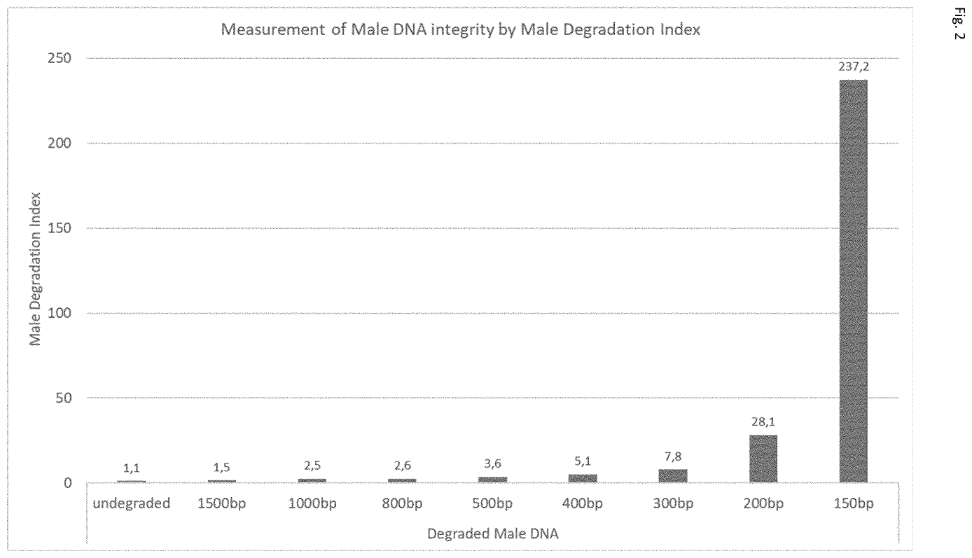

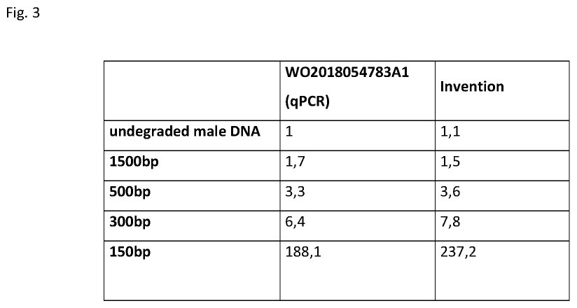

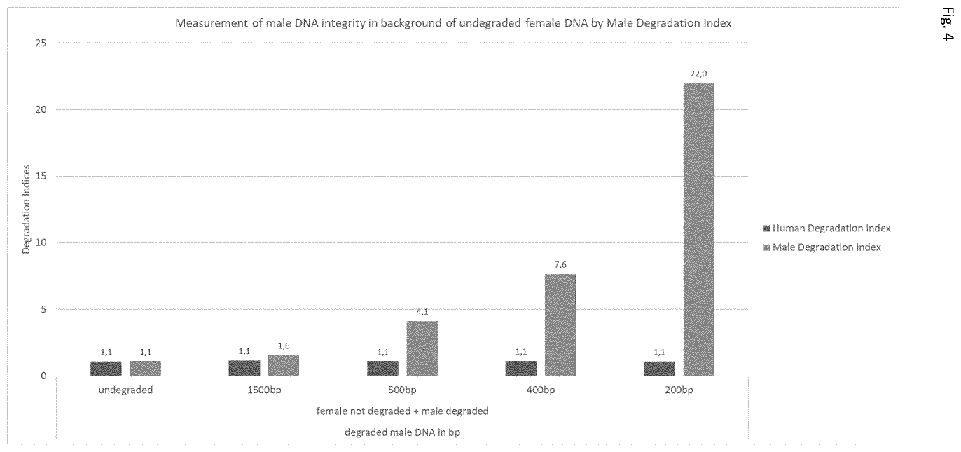

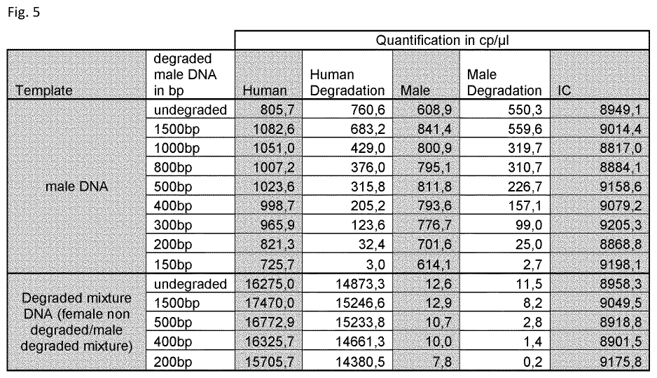

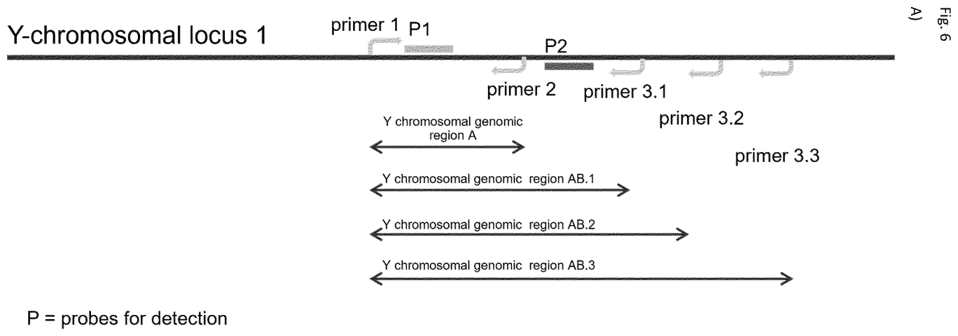

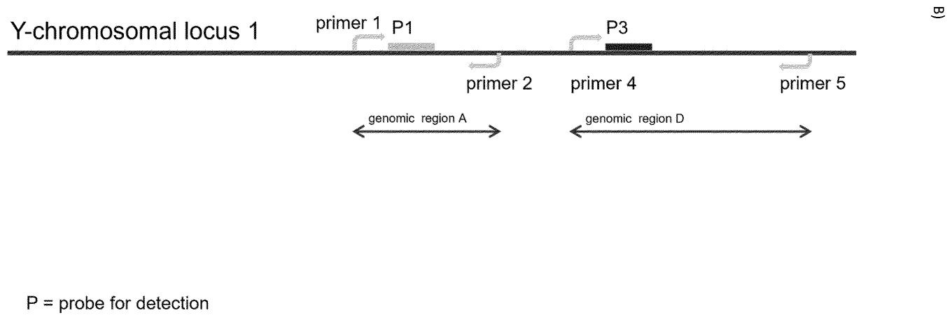

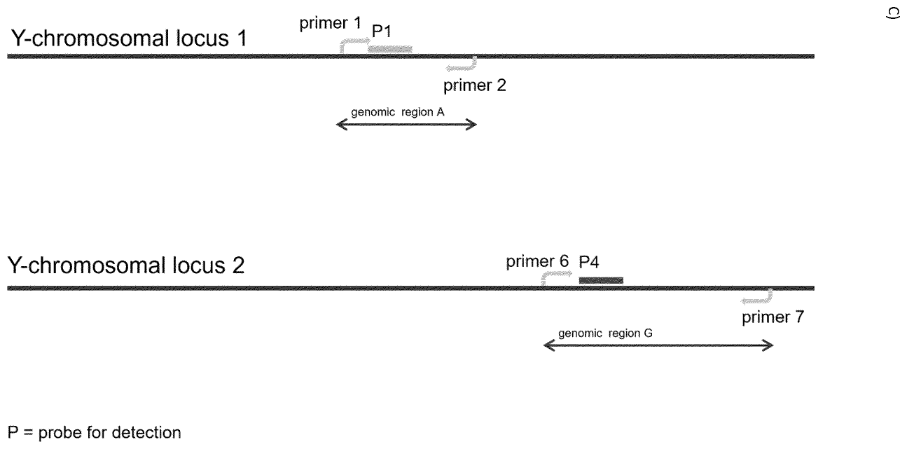

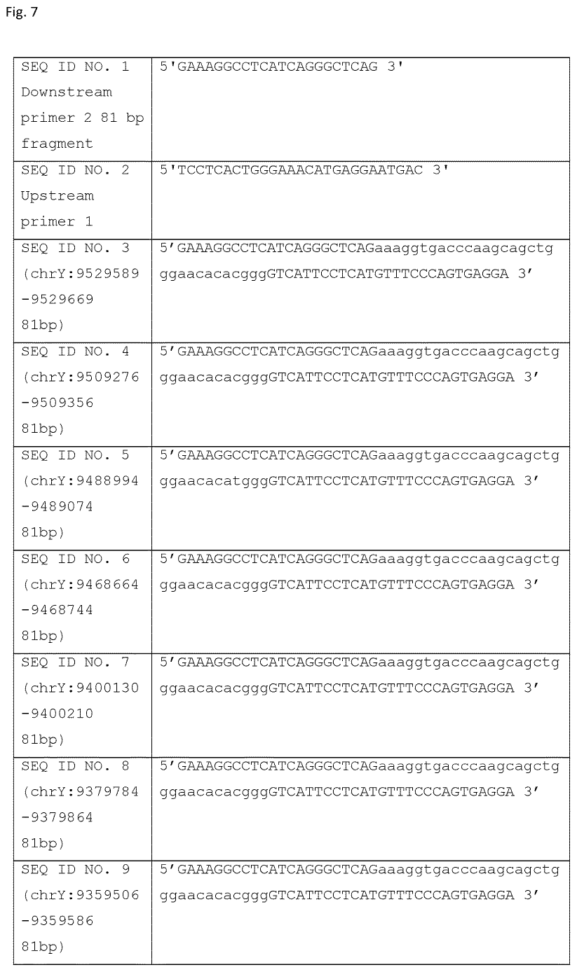

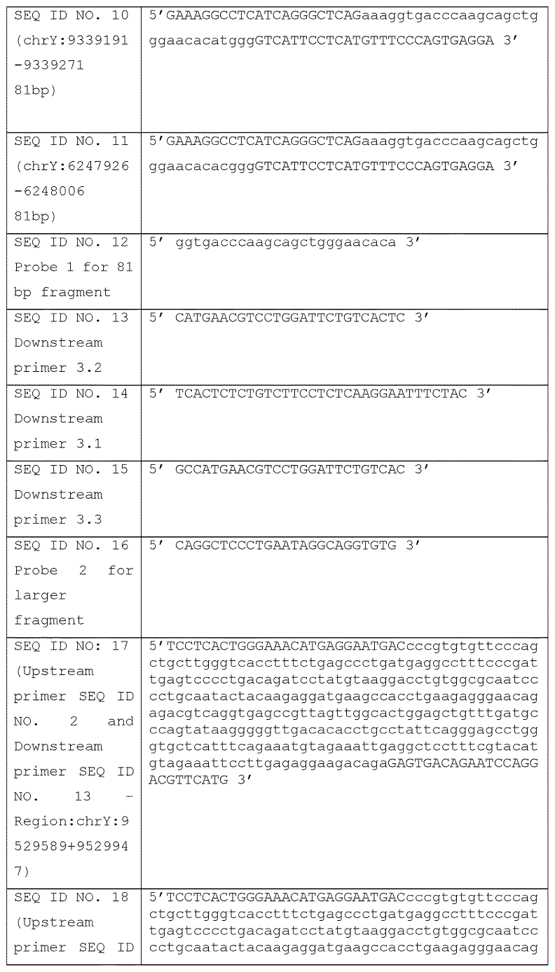

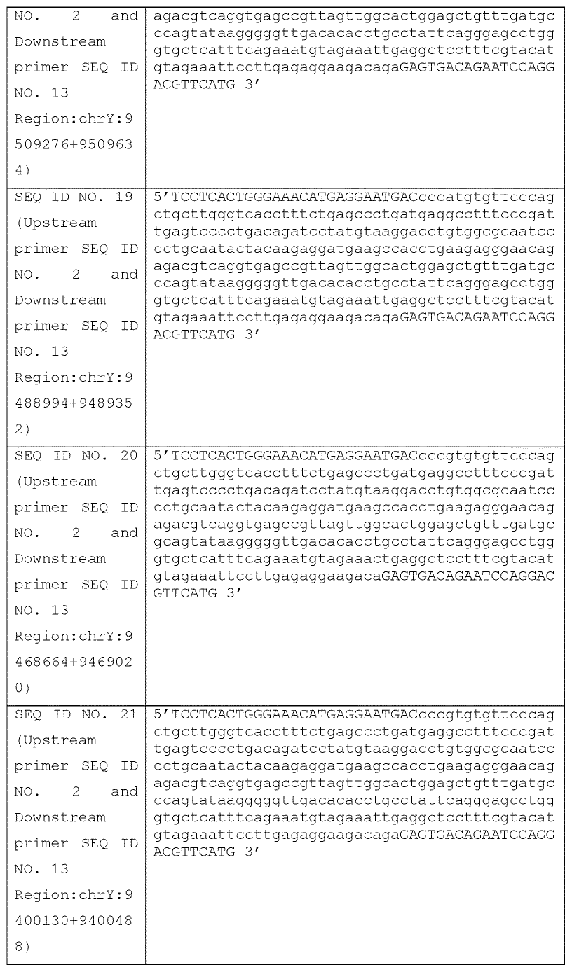

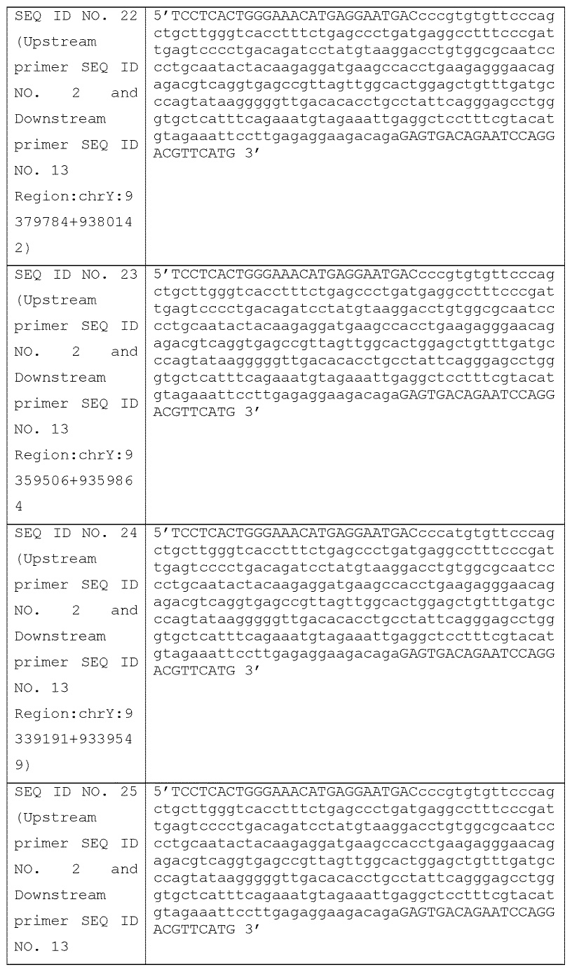

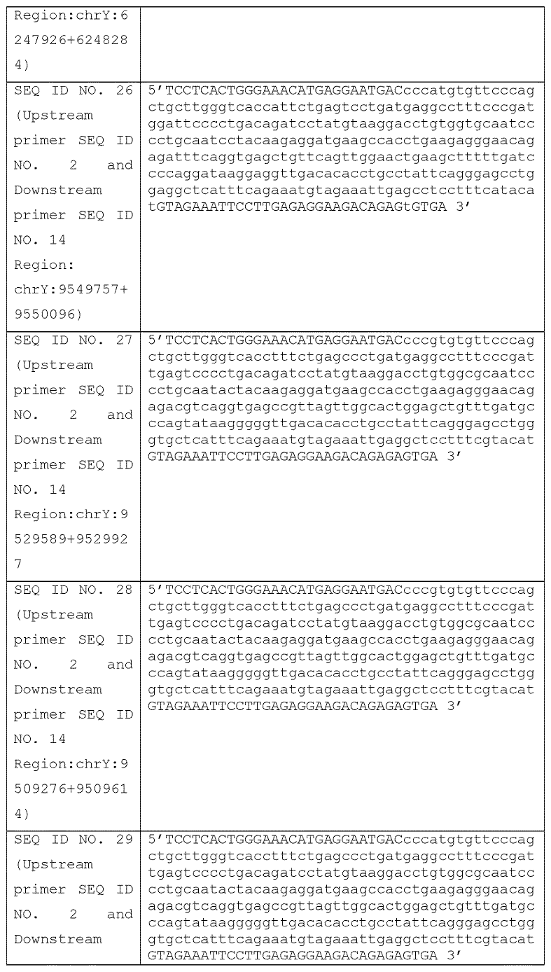

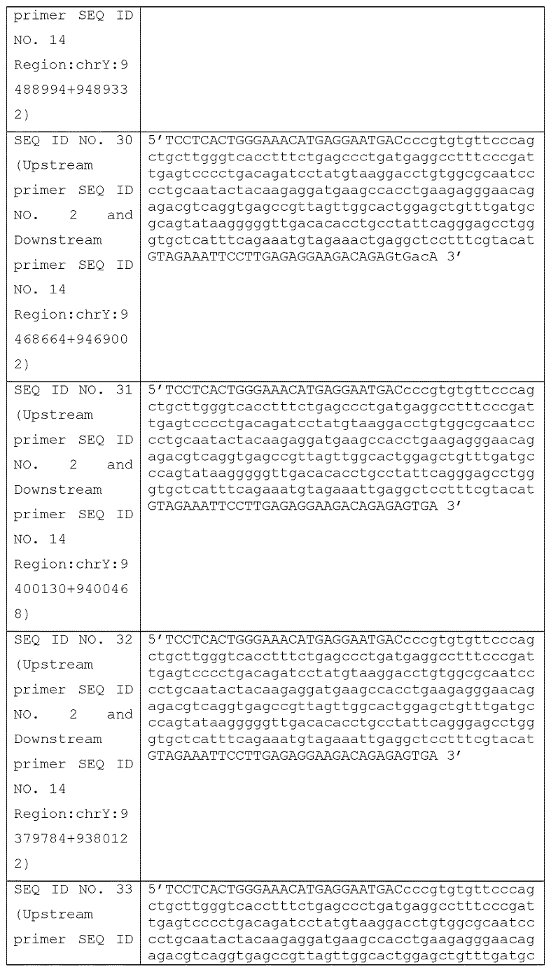

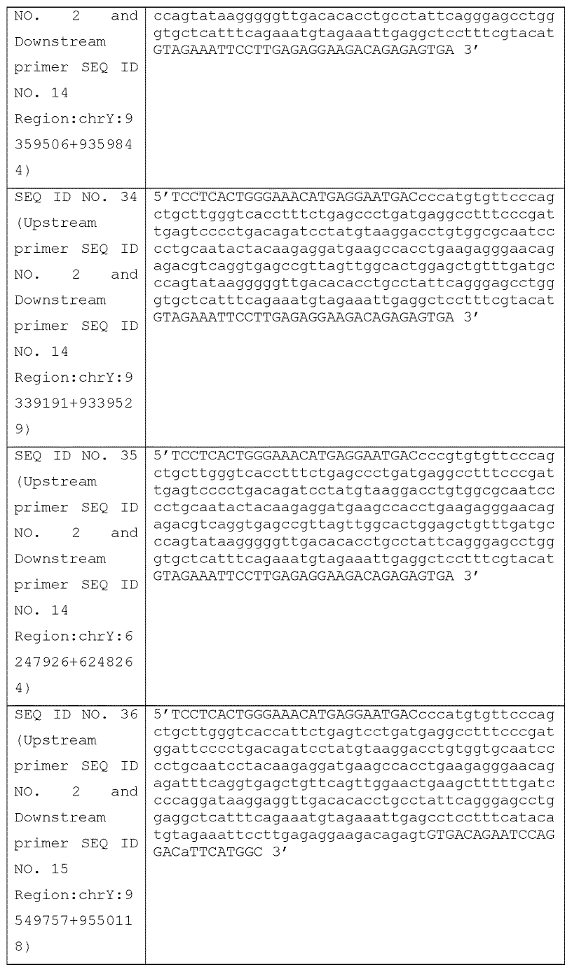

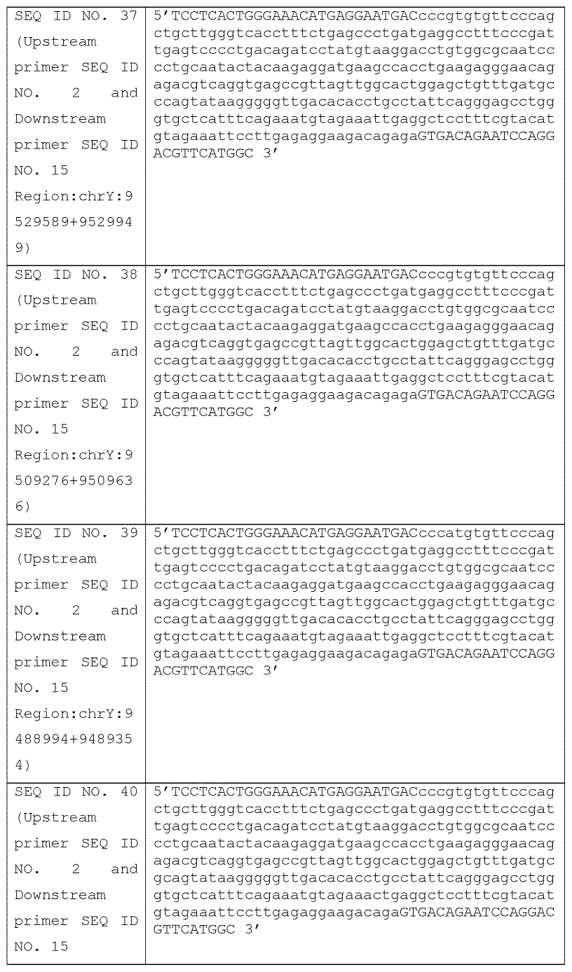

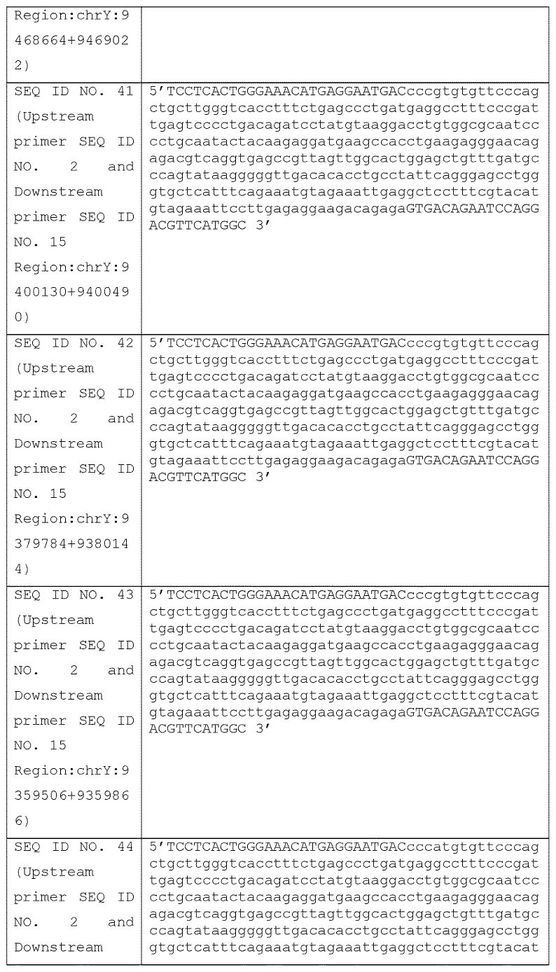

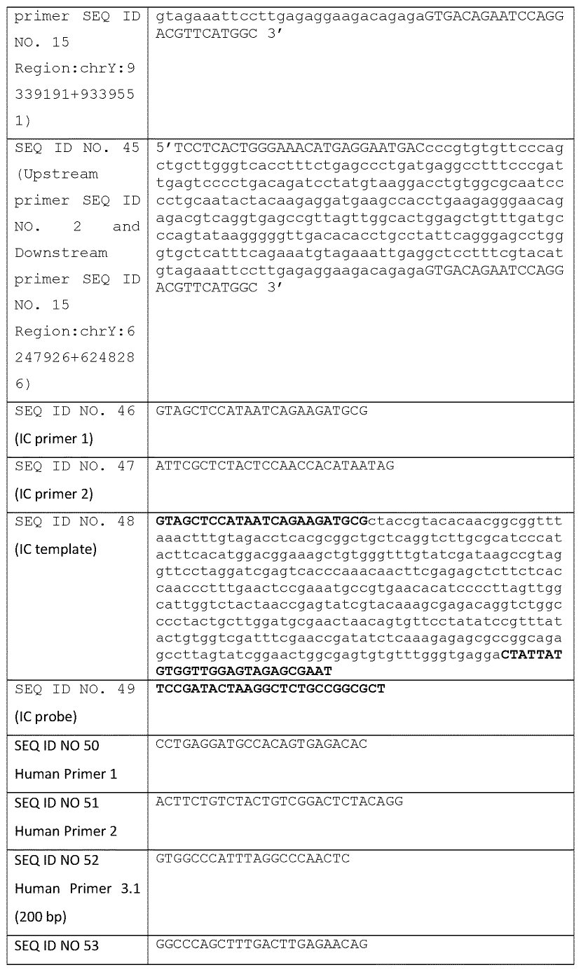

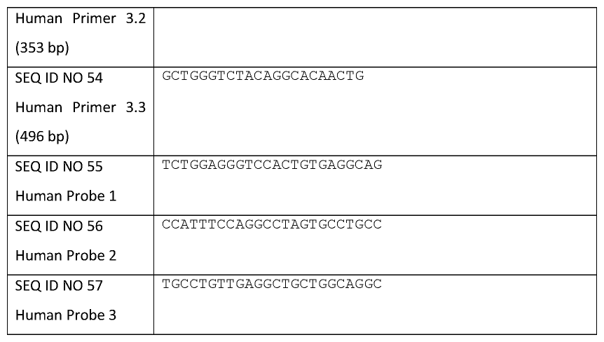

Example 5 and FIG. 5 shows multiplex amplification of human target, degradation target, male target, male degradation target and an internal amplification control. FIG. 5 shows multiplex amplification of human target, degradation target, male target, male degradation target and internal amplification control. A multiplex amplification setup according to the invention where 5 different targets (5-plex: human, degradation, male, male degradation and internal amplification control (IC)) have been successfully amplified in parallel. Noticeably, the method according is able to quantify human DNA, to assess human DNA integrity, to quantify male DNA and assess male DNA integrity and to report successful digital PCR amplification by the use of an internal amplification control in parallel.

Example 6 and FIG. 6 shows a schematic overview of Y-chromosomal DNA amplification for DNA integrity assessment. Depicted are schematic drawings showing possible amplification set-ups. FIG. 6A shows different combinations of primer pairs for the amplification of overlapping regions on a locus, e.g. a multicopy or single copy locus, located on the Y-chromosome.

FIG. 6B shows different combinations of primer pairs for the amplification of two non-overlapping regions on a locus located on the Y-chromosome.

FIG. 6C shows different combinations of primer pairs for the amplification of two regions located on two loci on the Y-chromosome.

FIG. 7 shows the sequences used as well as their numbering and names.

1-15. (canceled)

16. A method for detecting, quantifying, and determining the integrity and/or degradation status of male DNA in a sample comprising said male DNA, wherein the method comprises the steps of:
amplifying at least two regions on at least one locus within the Y-chromosome, wherein the amplification is done by digital PCR;
detecting the at least two amplification products through the use of at least two probes;
quantifying the amount of the at least two amplification products; and
determining the integrity and/or degradation status of the DNA of the sample by calculating a degradation index;
wherein at least one of the at least two probes binds to one of the at least two amplification products and at least one other probe of the at least two probes binds to another one of the at least two amplification products, and
wherein at least one of the at least two amplification products is longer than another one of the at least two amplification products.

17. The method according to claim 16, wherein the amplification step is performed using at least one primer selected from one of the groups consisting of:
SEQ ID NO. 1 and SEQ ID NO. 2;
a reverse complement of SEQ ID NO. 1 and SEQ ID NO. 2; and
a primer that shares at least 90% sequence identity with one of the primers with SEQ ID NO. 1 and SEQ ID NO. 2 or a reverse complement thereof.

18. The method according to claim 16, wherein the amplification step is performed using a primer pair selected from one of the groups consisting of:
SEQ ID NO. 1 and SEQ ID NO. 2;
a reverse complement of SEQ ID NO. 1 and SEQ ID NO. 2; and
a primer that shares at least 90% sequence identity with one of the primers with SEQ ID NO. 1 and SEQ ID NO. 2 or a reverse complement thereof.

19. The method according to claim 16, wherein the amplification is performed using a primer pair with a sequence according to SEQ ID NO. 1 and SEQ ID NO. 2.

20. The method according to claim 16, wherein said sample originates from whole blood, blood fractions, plasma, serum, an oral specimen, saliva, sputum, urine, human biopic tissue, clothing containing biological material, vaginal swabs, sperm, skin swabs, wound swabs, biological material or parts of the human body having an availability for isolation of a genome.

21. The method according to claim 16, wherein said sample comprises both male genomic DNA and female genomic DNA.

22. The method according to claim 16, wherein the at least two regions on the at least one locus within the Y-chromosome are not overlapping.

23. The method according to claim 16, wherein the at least two regions on the at least one locus within the Y-chromosome are overlapping.

24. The method according to claim 23, wherein the at least two overlapping regions on the at least one locus within the Y-chromosome are amplified using at least one common primer.

25. The method according to claim 16, wherein the at least one locus within the Y-chromosome is a single copy and/or a multicopy locus or at least one single copy locus and at least one multicopy locus (MCL-Y) within the Y-chromosome.

26. The method according to claim 16, wherein the amplification step further comprises the amplification of an internal amplification control (IC).

27. The method according to claim 16, wherein the at least two amplified regions are localized on at least two loci within the Y-chromosome.

28. The method according to claim 27, wherein the at least two loci within the Y-chromosome are single copy and/or multicopy loci or at least one single copy locus and at least one multicopy locus (MCL-Y) within the Y-chromosome.

29. The method according to claim 16, wherein the at least one locus within the Y-chromosome is a multicopy locus (MCL-Y).

30. The method according to claim 16, wherein said locus shares at least 85% sequence identity to a sequence according to SEQ ID NO. 3 over a stretch of at least 60 base pairs (bp) or with the reverse complement thereof.

31. The method according to claim 16, wherein the locus is amplifiable with a primer pair according to SEQ ID NO. 1 and 2 or the reverse complement thereof.

32. A kit for performing a method according to claim 16, wherein said kit comprises at least one primer selected from the group of SEQ ID NO 1, SEQ ID NO 2, SEQ ID NO 13, SEQ ID NO 14 and/or SEQ ID NO 15 and/or complements thereof.

33. The kit according to claim 32 further comprising at least one probe selected from the group of SEQ ID NO 12 and/or SEQ ID NO 16 and/or complements thereof.
